(19) United States
(12) Patent Application Publication
(10) Pub. No.: US 2015/0164567 A1
(43) Pub. Date: Jun. 18, 2015

(54) SURGICAL TOOL SYSTEM

(71) Applicant: UNIVERSITAT ZURICH, Zürich (CH)

(72) Inventor: Bernhard Dimitris Ciritsis, Zurich (CH)

(73) Assignee: UNIVERSITAT ZURICH, Zurich (CH)

(21) Appl. No.: 14/373,660

(22) PCT Filed: Jan. 23, 2013
(86) PCT No.: PCT/EP2013/051206

§ 371 Date: Jul. 22, 2014

(30) Foreign Application Priority Data
Jan. 23, 2012 (EP) 12152057.1

(51) Int. Cl. A61B 17/88; A61B 17/16

(52) U.S. Cl. CPC A61B 17/8805; A61B 17/1637; A61B 17/8811; A61B 17/8816

(57) ABSTRACT

The invention relates to a surgical tool for performing surgery on a bone, particularly for reinforcing the bone in a miniinvasive manner, comprising: a first and a second element (10, 20), wherein the two elements (10, 20) each comprise a shell (100, 200) defining a hollow space (101, 201) of the respective element (10, 20), wherein the second element (20) is designed to be inserted into the hollow space (101) of the first element (10), a guiding element (30), wherein the guiding element (30) comprises a first free end (31), and wherein the guiding element (30) is designed to be inserted into the hollow space (201) of the second element (20) with said first free end (31) of the guiding element (30) ahead, and wherein the guiding element (30) is designed to be drilled into said bone with its first free end (31) ahead so as to anchor said guiding element (31) in said bone, and a drill (40), which drill (40) is designed to be put over the guiding element (30) in a drilling state, in which drilling state the first element (10) encompasses said drill (40) for tissue protection.

The invention relates to a surgical tool for performing surgery on a bone, particularly for reinforcing the bone in a miniinvasive manner.

Osteoporotic fractures, especially in the femoral neck area are already a challenging surgical problem that will become even more important in the next future because of the growing percentage of elderly people in the western societies.

Already in 1990 there were more than 1.7 Million cases of femoral neck fractures worldwide. The life time incident rate is between 10% and 20%.

A study recently published by the WHO shows that the fracture of the femoral neck is one of the ten most severe pathologies worldwide, causing death in up to 25% of the patients in one year after the fracture. More than 50% of the patients will be chronically invalid after a fracture of the femoral neck and 25% need continuously nursing.

The risk of fracture of the controlateral side after a first femoral neck fracture is 6 to 9 times higher than the life time incident rate.

The socioeconomic impact for this pathology is enormous and still growing: In 2000, alone in Switzerland, the costs for acute hospital care were estimated to amount to more than 95 Million Swiss Francs.

Up to now, the prior art to resolve the pathology of femoral neck fractures is to realize an osteosynthesis or to replace the femoral head by a prosthesis. Both ways are, as mentioned above, linked to high costs and high morbidity and mortality.

Therefore, the problem underlying the present invention is to provide for a surgical tool that helps in preventing fracture due to osteoporosis, particularly in a miniinvasive way, meaning very low blood loss and a short time of surgical intervention.

This problem is solved by a surgical tool having the features of claim 1.

According thereto, the surgical tool comprises: a first and a second element each extending along a longitudinal axis, wherein the two elements each comprise a shell encompassing a hollow space of the respective element, wherein the second element is designed to be inserted into the hollow space of the first element in order to arrange the two elements in an embedded state, in which the two elements are arranged coaxially with respect to each other and in which the shell of the first element encompasses the shell of the second element, wherein particularly the second element being arranged in said embedded state is designed to be pushed with a first free end of the second element ahead through pre-incised tissue towards a bone to be reinforced so as to make cortical contact with said bone; a guiding element (e.g. guiding wire) extending along a longitudinal axis, wherein the guiding element comprises a first free end, and wherein the guiding element is designed to be inserted into the hollow space of the second element with said first free end ahead in order to arrange the guiding element in an inserted state with respect to the second element when the latter is arranged in said embedded state, in which inserted state the guiding element is arranged coaxially with respect to the second element and encompassed by the shell of the second element, and wherein the guiding element is designed to be drilled into a bone to be treated with its first free end ahead when being arranged in said inserted state so as to fasten said guiding element to said bone; and a drill extending along a longitudinal axis, which drill comprises a shell encompassing a hollow space of the drill, wherein the drill, particularly after having removed the second element from the first element and from the guiding element, is designed to be put over the guiding element with a drill head ahead in order to arrange the drill in a drilling state with respect to the guiding element and the first element, in which the first element encompasses said drill for tissue protection and in which the guiding element is arranged in the hollow space of the drill so as to guide said drill upon drilling, and wherein the drill head is designed to drill a borehole into the corticalis and the spongiosa of said bone in said drilling state.

Since the afore-described surgical tool comprises a number of separate components interacting with each other one may also speak of a surgical tool system.

Advantageously, the tool according to the invention particularly allows for getting access to the spongiosa of a bone, particularly the femur, in a defined and precise manner, thus laying the foundation for reinforcing the bone in order to prevent it from fracture.

Further, the tool according to the invention can be used to realize (X-ray assisted) biopsy out of an e.g. tumerous bone or to get access to another kind of osteolytic zone and to reinforce it, even to place medicaments into a bone.

The tool according to the invention is particularly suited for the femoral neck but can also be used for other (big) bones in the human body.

Preferably, the first and second element, the guiding element, as well as the drill (and other elements described below) are made of (e.g. surgical) steel.

Further, the shells of the first and the second element as well as the shell of the drill are particularly formed (at least in sections) hollow circular cylindrical. Preferably, the guiding element is formed at least in sections as a circular cylinder. Thus, said longitudinal axes of the afore-described elements are preferably cylinder axes. Correspondingly, the hollow spaces are shaped (at least in sections) cylindrical. Further, at the first free end of the guiding element, a drill head is particularly provided for drilling the guiding element into the bone, wherein particularly said drill head may be tapered towards the first free end of the guiding element.

Preferably, the second element, particularly after removal of the drill and the first element from the guiding element and re-arrangement of the second element over the guiding element such that the latter is arranged in the hollow space of the second element, is designed to be inserted with its free end into said borehole (i.e. into a corticalis section of said borehole drilled by means of said drill), particularly so as to bridge the corticalis of said bone, i.e., the spongiosa lying beneath the corticalis of said bone is then (directly) accessible via the hollow space of the second element.

Further, the tool according to the invention further comprises a cannula (also made of (surgical) steel, preferably), which cannula extends along a longitudinal axis, wherein the cannula comprises a shell encompassing a hollow space of the cannula, through which bone cement can be piped that is to be injected into said borehole, wherein the cannula is designed to be inserted into the hollow space of the second element (for serving as a guiding means for the cannula) with a first free end of the cannula ahead in order to arrange the cannula in an injection state with respect to the second element, particularly after the guiding element has been removed from the second element, in which injection state the cannula is arranged coaxially with respect to the second element and encompassed by the shell of the second element.

Preferably also the cannula is formed as a hollow circular cylinder such that its longitudinal axis is a cylinder axis. Correspondingly the hollow space of the cannula is formed cylindrical in particular. The cannula may be single use.

Preferably, the second element can be inserted into the first element in a form fitting manner. Particularly, a slight play can be present between the two engaged elements that is needed for being able to easily slide the second element back and forth with respect to the first element manually. Likewise, the cannula is preferably insertable into the hollow space of the second element in an essentially form-fitting manner, the guiding element is preferably insertable into the hollow space of the second element or of the drill in an essentially form-fitting manner, the drill is preferably insertable in the hollow space of the first element in an essentially form-fitting manner. A typical play may have a range between 0.0 mm (no play at all) and 0.5 mm.

Further, the cannula is preferably designed to be inserted into the second element such that it projects with its first free end past the hollow space of the second element (i.e. out of an opening delimited by the first free end of the second element), particularly through said borehole into the spongiosa of the bone, wherein particularly said first free end of the cannula delimits an injection opening of the cannula, through which bone cement can be discharged out of the hollow space of the cannula into the borehole, i.e., through the corticalis into the spongiosa in order to generate a bone cement implant reinforcing the bone. The cannula is further designed to be movable inside the hollow space of the second element, so that it can be retracted upon injecting bone cement into the spongiosa.

Particularly, the tool further comprises a stamper, particularly made from (surgical) steel, extending along a longitudinal axis, wherein the stamper is designed to be inserted into the hollow space of the cannula with a first free end of the stamper ahead, particularly in a form-fitting manner, in order to push the remaining bone cement by means of said first free end out of the hollow space of the cannula, particularly through said borehole into the spongiosa of the bone. The stamper may be single use.

Particularly, the stamper being inserted in the hollow space of the cannula and the cannula are designed to be pushed together along their coaxial longitudinal axes in the hollow space of the (stationary) second element, wherein said second element is designed to guide the cannula and the stamper upon said movement, particularly so as to push bone cement injected into said borehole into the spongiosa, thereby forming a circular hollow cylindrical bone cement implant in the spongiosa of said bone.

It is to be noted that this creates a lightweight implant having a higher resistance with respect to a bending load compared to a full cylinder due to the essentially hollow cylindrical form of the implant created by said perforation of the injected bone cement.

The tool system can be adapted such concerning its dimensions that the hollow cylindrical bone cement implant comprises the desired dimensions concerning for instance the inner diameter of its hollow space or its wall thickness.

Preferably, the stamper comprises a second free end opposing the first free end along the longitudinal axis of the stamper, wherein the stamper comprises a circular plate (head) at the second free end extending perpendicular to the longitudinal axis of the stamper, from which broadened head a circular cylindrical shaft of the stamper protrudes forming the portion of the stamper that can be inserted into the hollow space of the cannula. Thus, the longitudinal axis of the stamper is also a cylinder axis in particular.

Further, when the second element is (initially) in its embedded state with respect to the first element, the first free end of the second element particularly protrudes out of the first element along its longitudinal axis (i.e. out of an opening of the first element delimited by the first free end of the first element) for making cortical contact, wherein said first free end of the second element is preferably rounded. Preferably, also the first free end of the first element is rounded to reduce the risk of injury.

Particularly, the second element comprises a second free end opposing the first free end of the second element along the longitudinal axis of the second element, wherein particularly said second free end circulates (i.e. extends circumferentially around) an opening of the second element through which said guiding element and said cannula can be inserted into the hollow space of the second element.

Preferably, the second element comprises a (free end) portion near the second free end that broadens towards said second free end, particularly such that said portion comprises at least in sections the shape of a truncated cone, wherein the second free end of the second element preferably forms a circulating (e.g. ring-like or circumferentially extending) protrusion protruding from an outside of said portion.

Particularly, also the first element comprises a second free end opposing the first free end of the first element along its longitudinal axis, wherein particularly said second free end circulates (i.e. extends circumferentially around) an opening of the first element, through which the second element and said drill can be inserted into the hollow space of the first element.

For receiving the tapered portion of the second element, the hollow space of the first element preferably comprises a section along which the hollow space broadens towards said opening (second free end) of the first element, particularly such that said section of the hollow space also comprises the shape of a truncated cone.

Also here, the second free end of the first element particularly forms a circulating (e.g. ring-like or circumferentially extending) protrusion protruding from an outside of the shell of the first element.

Since the drill is a hollow member, its drill head encompasses an end portion of the hollow space of the drill. Preferably, for cutting the bone and banking of the borings upon drilling, the drill head is particularly formed as a spiral drill.

In order to be able to determine the current depth of a borehole drilled with the drill upon drilling as well as to support banking of the borings, the shell of the drill preferably comprises an outside, on which a plurality of recesses are formed extending at least in section along a circumferential direction of the drill (i.e. across the longitudinal axis of the drill), which recesses (grooves) are equidistantly distributed along the longitudinal axis of the drill.

Further, in order to be able to couple a second free end of the drill opposing the drill head of the drill along the longitudinal axis of the drill to an actuator (e.g. drilling machine) for rotating the drill about its longitudinal axis, the drill preferably comprises a hexagonal cross sectional contour at the second free end so that it may be releasably fixed in a boring socket of said actuator.

Preferably, the tool further comprises a closure element being designed to be introduced into said borehole, particularly through the cannula by means of the stamper, particularly so as to retain bone cement that was injected into the borehole (e.g. to prevent the bone cement from dripping out of the borehole).

Particularly, the closure element is designed to be introduced into said borehole in a folded state, wherein the closure element is further designed to unfold at least partially when positioned in said borehole, particularly such that the closure element butts against a surrounding wall of said borehole. Preferably, an outer diameter of the closure element then corresponds to the outer diameter of the cannula or inner diameter of the borehole.

Further, the closure element preferably comprises an outer contour which comprises a serrated shape in said folded state and preferably an octagonal shape in a completely unfolded (e.g. flat) state.

Preferably, the closure element is made out of a material or comprises a material, particularly paper, particularly sterilized Lackmus paper, allowing for detecting the closure element by means of electromagnetic radiation, particularly X-rays, wherein particularly said paper or material is dyed, particularly in standardized contrast liquid solution, so that is visible under X-ray control.

Further, the problem according to the invention is solved by a method for treating, examining and or reinforcing a bone, which may comprise the following successive steps and may use the surgical tool according to the invention.

Particularly, a patient with diagnosed osteoporosis and/or high risk of fracture of the femoral neck or another (big) bone is installed on an extension table without traction in a supine position.

Particularly, the position of the femoral neck (or another region of a bone) and the point of skin incision is controlled and validated, particularly by means of x-ray, particularly in an antero-posterior (a.p.) and/or in a lateral view.

Particularly, a small skin incision is realized over the chosen point.

Particularly, soft tissues are split by introducing a second element (also denoted as canulated candle), which again is embedded in a first element (also denoted as jacket).

Particularly, the second element together with the first element is pushed forward until there is cortical contact with the bone to be examined, treated and/or reinforced.

Particularly, the right position of said elements is controlled, particularly under x-ray.

Particularly, a guiding element (e.g. guiding wire) is drilled into the bone (e.g. femoral neck) through the second element (canulated candle).

Particularly, the right position of the guiding element is controlled, particularly under x-ray.

Particularly, the second element is extracted from the first element.

Particularly, a drill (being canulated, i.e., comprising a hollow space) is introduced over the guiding element, so as to drill a hole into the corticalis and the spongiosa of the bone (e.g. femoral neck), particularly under x-ray control. Particularly, the first element is kept in place to protect soft tissues while drilling.

Particularly, the drill is taken out of the bone, the guiding element is kept in place, and the first element is removed.

Particularly, the second element is reintroduced over the guiding element and softly tapped into the drilled hole of the corticalis of the bone (e.g. femoral neck area) featured by the drill until the corticalis is bridged by the second element.

Particularly, the position of the second element is controlled, particularly by means of x-ray, and the guiding element is taken out of the bone (e.g. femoral neck).

Particularly, a cannula is introduced through the second element, and bone cement is injected, particularly under x-ray control, into the drilled hole in the bone (e.g. femoral neck area) through said cannula.

Particularly, while injecting the bone cement, the cannula is slowly retracted, particularly under x-ray control, from the drilled hole until reaching the edge (first free end) of the second element, still fixed in the corticalis of the bone (e.g. femoral neck).

Particularly, the cannula is kept in place and a stamper, particularly of equal length, is introduced into the cannula to push the remaining bone cement in the cannula forward into the bone.

Particularly, the bone cement is perforated, particularly under x-ray control, by tapping the stamper together with the cannula gently into the bone cement implant positioned in the bone (e.g. femoral neck area) so that the bone cement is pushed into the spongiosa.

Particularly, the stamper together with the cannula is removed from the bone cement when it begins to dry while the second element is kept in place.

Particularly, the second element is removed from the corticalis of the bone (e.g. femoral neck) when the bone cement is completely dry. Particularly, the surgical field is washed out and the skin incision is closed. Particularly, a last (e.g. x-ray) control, particularly in a.p. and lateral view, is performed.

Particularly, before injecting the bone cement into the borehole a closure element is introduced into said borehole, particularly so as to retain bone cement that is to injected into the borehole. This is preferably done in case the drilled hole done by the guiding element breaks through the bone in order to prevent bone cement from dropping out of the borehole.

Particularly, the closure element is introduced into said borehole in a folded state, wherein the closure element unfolds at least partially when positioned in said borehole, particularly such that the closure element presses against a surrounding wall of said borehole or hole drilled by means of the guiding element.

Further, the closure element is preferably pushed into the borehole through the cannula with help of the stamper, particularly under visualization of the closure element by means of electromagnetic radiation, particularly X-rays, before introducing bone cement into the borehole.

Therefore, particularly, the closure element is designed to be detected by means of X-rays.

Further features and advantages of the invention shall be described by means of a detailed description of an embodiment with reference to the Figures, wherein

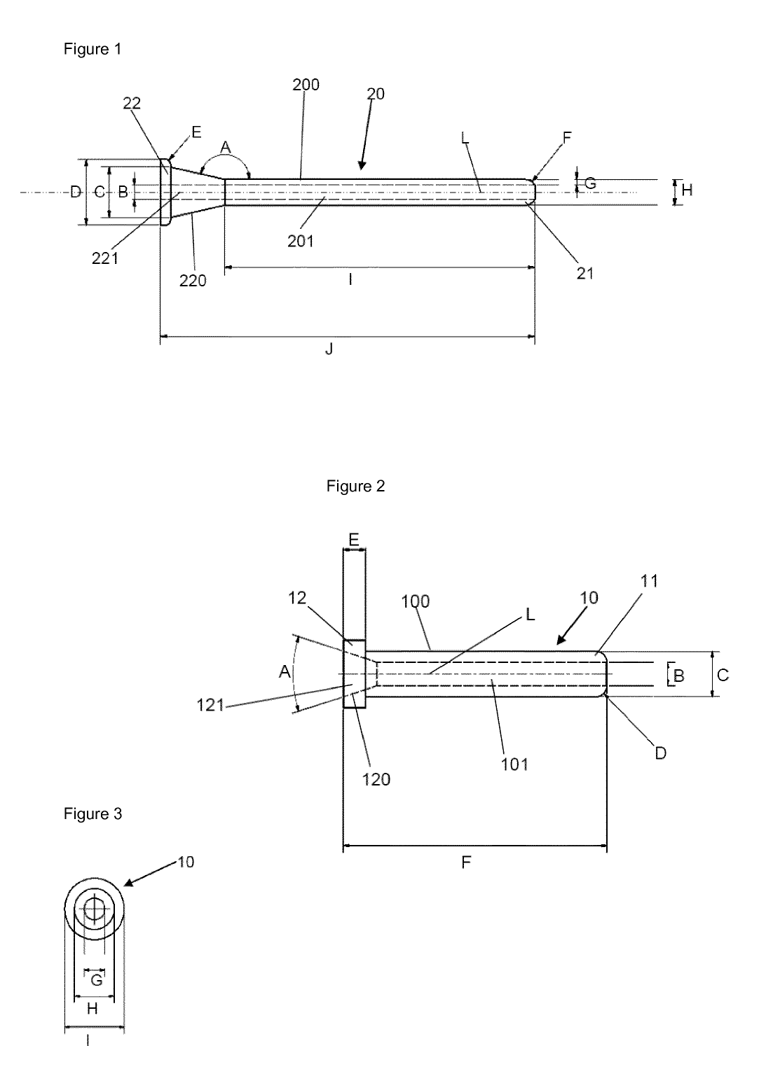

FIG. 1 shows a cross sectional view of a second element (canulated candle) of a tool according to the invention,

FIG. 2 shows a cross sectional view of a first element (metal jacket) of the tool according to the invention,

FIG. 3 shows a cross sectional view of the first element shown in FIG. 2 in a plane perpendicular to the longitudinal axis of the first element,

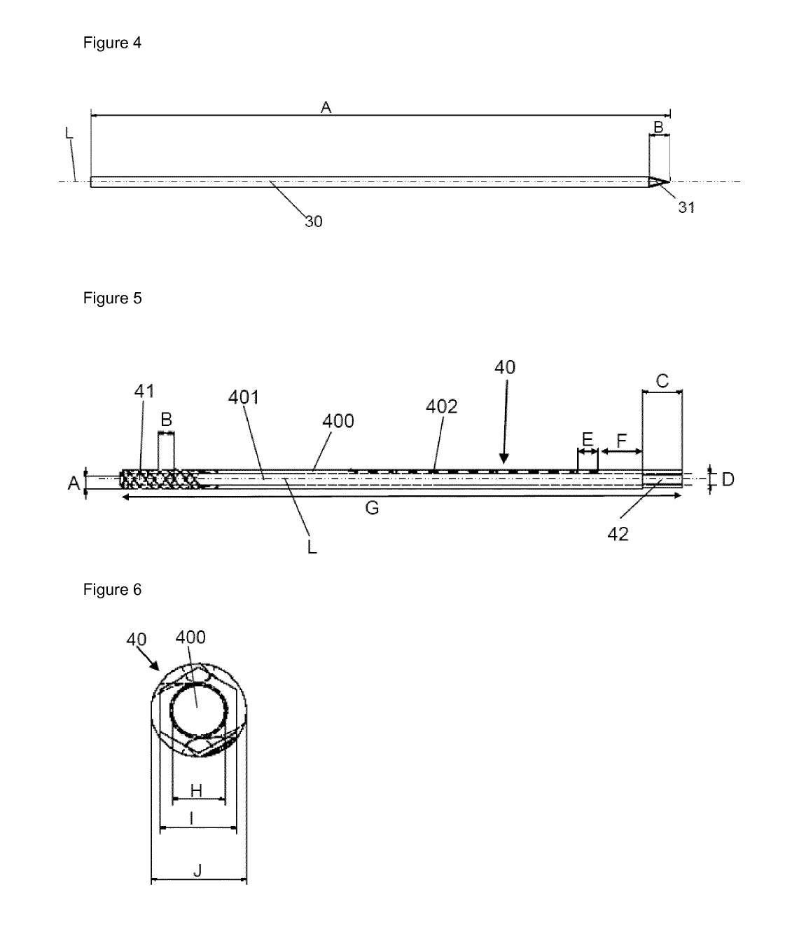

FIG. 4 shows a cross sectional view of a guiding element of the tool according to the invention,

FIG. 5 shows a cross sectional view of a drill of the tool according to the invention,

FIG. 6 shows a cross sectional view of the drill shown in FIG. 5 in a plane perpendicular to the longitudinal axis of the drill,

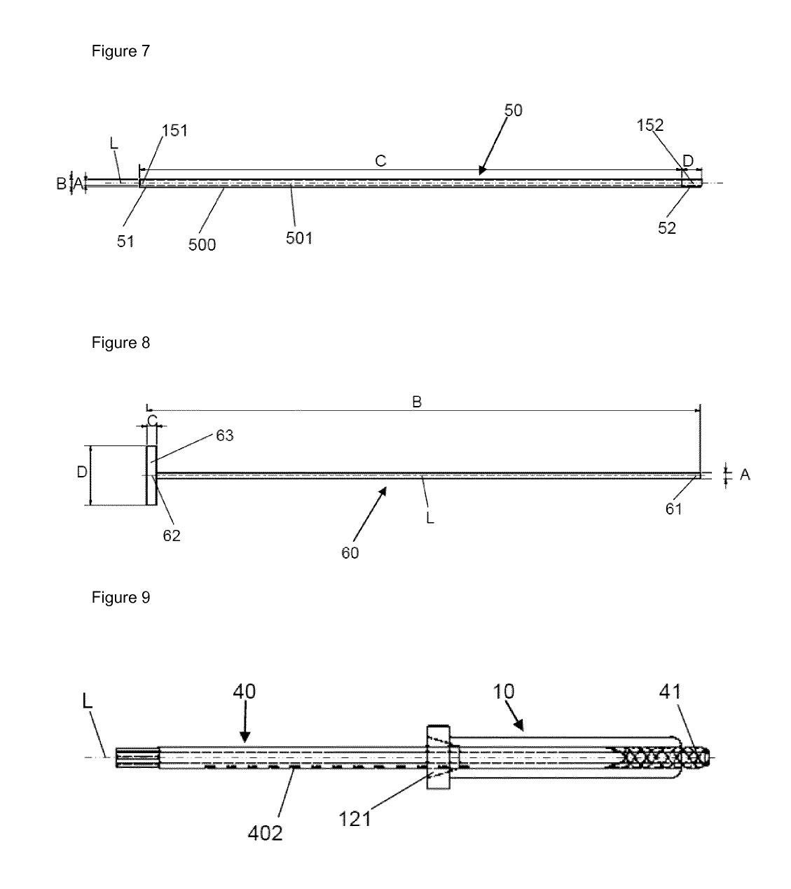

FIG. 7 shows a cross sectional view of a cannula of the tool according to the invention

FIG. 8 shows a cross sectional view of a stamper of the tool according to the invention,

FIG. 9 shows a cross sectional view of the drill inserted into the first element,

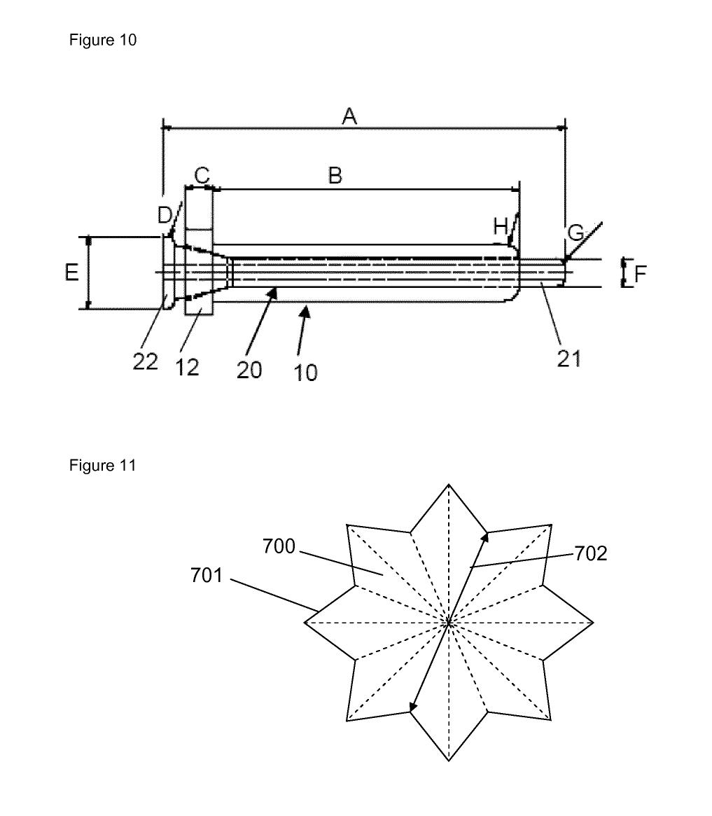

FIG. 10 shows a cross sectional view of the second element being inserted into the first element, and

FIG. 11 shows a plan view of a closure element for closing a drilled hole.

FIGS. 1 to 10 show the separate components of a tool according to the invention.

Said tool comprises an elongated first element (metal jacket) 10 as shown in FIGS. 2 and 3 that comprises a shell 100 surrounding an elongated hollow space 101. The shell 100 and the hollow space 101 each extend along a longitudinal (cylinder) axis L of the first element 10.

The first element 10 comprises a first free end 11 that is rounded (curvature radius D), as well as an opposing second free end 12 that forms a circular protrusion of the first element 10. Further, for simplifying insertion of a second element 20 into the hollow space 101 of the first element 10, the hollow space 101 of the first element 10 comprises a section 120 encompassed by said protrusion 12 that broadens towards an opening 121 delimited by said protrusion (second free end) 12 in the form of a truncated cone.

The first element 10 serves for protecting tissue covering a bone that is to be reinforced by means of the tool according to the invention.

The dimensions of the first element 10 may be chosen according to FIGS. 2 and 3 to be A=35°, B=10.50 mm, C=20.00 mm, curvature D=5.00 mm, E=10.00 mm, F=120.00 mm, G=10.50 mm, H=20.00 mm, and I=30.00 mm, in a second embodiment of the surgical tool F may be different, e.g. F=100.00 mm, while the other dimensions A, B, C, D, E, G, H, and I of the first element 10 are preferably not changed.

The tool further comprises a second element 20 shown in FIG. 1 that is to be housed by the first element 10 in an embedded state as shown in FIG. 9. The second element 20 also extends along a longitudinal (cylinder) axis L and comprises a shell 200 surrounding an elongated hollow space 201, too.

The second element (canulated candle) 20 is designed to be introduced into the hollow space 101 of the first element 10 with a first free end 21 of the second element 20 ahead through said opening 121 of the first element 10, wherein, when the second element 20 is fully inserted into the hollow space 101 of the first element 10, said first free end 21 of the second element 20 protrudes out of the hollow space 101 of the first element 10 through the first free end 11 of the first element 10 along its longitudinal axis L, thus providing a region of the second element 20 that serves for making cortical contact to the bone.

The second element 20 further comprises a second free end 22 opposing its first free end 21 along the longitudinal axis L of the second element 20 forming a circulating (e.g. circumferentially extending) protrusion 22 of the second element 20, which delimits an opening 221 of the second element 20 through which the hollow space 201 of the second element 20 is accessible. The shell 200 of the second element 20 further comprises a portion 220 that broadens towards said protrusion 22 in the form of a truncated cone, wherein said portion 220 is adapted to be received by the afore-described section 120 of the hollow space 101 of the first element, which forms a stop for the second element 20 (c.f. FIG. 10).

The dimensions of the second element 20 may be chosen according to FIG. 1 to be A=167°, B=5.50 mm, C=19.50 mm, D=25.50 mm, curvature E=2.00 mm, curvature F=4.50 mm, G=2.25 mm, H=10.00 mm, I=120.00 mm, and J=145.00 mm. In the second embodiment I and J may be different, e.g. I=100.00 mm and J=125.00 mm, while the other dimensions A, B, C, D, E, F, G, and H of the second element 20 are preferably not changed

With the two elements 10, 20 being arranged in the afore-described embedded state, an elongated guiding element 30 of the tool according to the invention as shown in FIG. 4 is provided, which guiding element 30 is essentially cylindrical, extends along a longitudinal (cylinder) axis L, and is designed to be inserted with a tapering drill head 31 ahead into the hollow space 201 of the second element 20 via the opening 221 of the latter, which hollow space 201 guides the guiding element 30 towards the bone. Then, the guiding element 30 is drilled into the bone in order to anchor it thereabouts.

The dimensions of the guiding element 30 according to FIG. 4 may be chosen to be A=280.00 mm, and B=10.00 mm. The drill head may comprise an outer tapered Whitworth thread (e.g. of specification R1/8-28 according to Din 2999).

After having anchored the guiding element 30, the second element 20 is removed from the first element 10, and the guiding element 30 now provides guidance for a further component of the tool according to the invention in the form of an elongated drill 40 as shown in FIGS. 5 and 6. The drill 40 comprises an essentially cylindrical shell 400 surrounding a hollow space 401 of the drill 40 extending along a longitudinal (cylinder) axis L. The drill 40 further comprises a drill head 41 at a first free end of the drill encompassing a free end portion of the hollow space 401 of the drill 40 as well as a second free end 42 opposing the drill head 41 along said longitudinal axis L, which second free end comprises a hexagonal shape in a cross section perpendicular to the longitudinal axis for clamping the drill 40 in a corresponding socket of an actuator (drilling machine, not shown).

For drilling a borehole into the corticalis and the spongiosa of the bone (e.g. femural neck), the drill 40 is inserted through the opening 121 of the first element 10, so as to arrange the drill 40 in the hollow space 101 of the first element 10, whereby the guiding element 30 is arranged in the hollow space 401 of the drill 40 to guide the latter upon drilling. At the same time, the first element 10 provides protection for the surrounding tissue. The drill 40 comprises a length along the longitudinal axis L such that the drill 40 protrudes out of the first element 10 with its drill head 41 at the first free end 11 of the first element 10 as shown in FIG. 9 (here, guiding element is not shown).

In order to control the depth of a borehole drilled by means of the drill 40 along the longitudinal axis L, the drill 40 further comprises equidistant recesses 402 on its shell 400 that also support banking of the borings.

The dimensions of the drill 40 may be chosen according to FIGS. 5 and 6 to be A=6.39 mm, B=7.64 mm, C=20.00 mm, D=5.50 mm, E=11.00 mm, F=22.00 mm, G=280.00 mm, H=5.50 mm, I=9.00 mm, and J=10.00 mm. In the second embodiment I may be different, e.g. I=8.00 mm, while dimensions A, B, C, D, E, F, G, H, and J of drill 40 are preferably not changed. Please note, that the dimensions the drill 40 may be adapted to a specific drill chuck of a drilling machine that is employed for drilling the respective borehole.

After having drilled a borehole into the corticalis and spongiosa of the bone, the drill 40 is taken out of the bone, whereas the guiding element 30 is kept in place. The first element 10 is also removed.

Now, the second element 20 is reintroduced over the guiding element 30 and pressed into the drilled borehole of the corticalis of the bone (femoral neck area) provided by the drill 40 in beforehand until the corticalis is bridged by the (first free end of the) second element 20.

Once the second element 20 is in place (i.e., properly inserted in the borehole), the guiding element 30 is taken out of the bone (femoral neck) and an elongated cannula 50 as shown in FIG. 7 comprising a shell 500 surrounding a hollow space 501 of the cannula 50 extending along a longitudinal axis L is introduced with a first free end 51 ahead in the hollow space 201 of the second element 20, which guides the cannula 50 towards the spongiosa into which bone cement is injected through the hollow space 501 of the cannula 50 via an injection opening 151 at the first free end 51 of the cannula 50. While injecting the bone cement, the cannula 50 is retracted until it reaches the first free end 11 of the second element 20 being anchored in the corticalis part of the borehole (in the femoral neck). Further, the cannula 50 comprises a second free end 52 having an outer thread (e.g. of specification M5x0.8-6g), which second free end 52 delimits an opening 152 of the cannula 50, through which the hollow space 501 of the cannula 50 is accessible.

According to FIG. 7, the cannula 50 may comprise the dimensions A=3.00 mm, B=5.00 mm, C=270.00 mm, and D=10.00 mm. In the second embodiment C may be different, e.g. C=220.00 mm, while the other dimensions A, B, and D of the cannula 50 are preferably not changed. Reducing the length of the cannula 50 allows for reducing the pressure of the bone cement in the cannula 50 while injecting it.

The cannula 50 is kept in place and an elongated stamper 60 according to FIG. 8 is inserted form-fittedly through said opening 152 of the cannula 50 into the hollow space 501 of the cannula 50 with a first free end 61 of the stamper 60 ahead. The stamper 60 further comprises an elongated cylindrical shaft extending along a longitudinal (cylinder) axis L, as well as a broadened head in the form of a circular plate 63 at a second free end 62 of the stamper 60 opposing the first free end 61 along the longitudinal axis L.

With the first free end 61 being inserted in the hollow space 501 of the cannula 50, remaining bone cement residing in said cannula 50 can be easily forwarded into the bone.

According to FIG. 8, the stamper 60 may comprise the dimensions A=3.00 mm, B=280.00 mm, C=5.00 mm, and D=30.00 mm. In the second embodiment, B may be different, e.g. B=230.00 mm, while dimensions A, C, and D of stamper 60 are preferably not changed.

Finally, the bone cement injected into the spongiosa is pushed laterally into the spongiosa by pushing the stamper 60 together with the cannula 50 into the injected bone cement implant positioned in the bone (e.g. femoral neck area) so that the bone cement implant assumes a hollow cylindrical shape particularly providing resistance against bending loads.

The stamper 60 together with the cannula 50 are removed from the bone cement when it begins to dry, the second element 20 is kept in place.

Once the bone cement is completely dry, also the second element 20 is removed from the corticalis of the bone (femoral neck).

In case, for instance, the drilled hole done by the guiding element 30 touches the articulation surface of the femoro-acetubular joint so that the borehole becomes a through-hole through the bone (e.g. femur), a closure element 700 according FIG. 11, particularly single use, is placed into the borehole (e.g. of the femoral neck), before the introduction of the bone cement to prevent bone cement from dropping out of the borehole on the other side (e.g. into the femoro-acetubular articulation). Preferably, the closure element 700 has a serrated outer contour 701 in a folded state shown in FIG. 11 (creases are shown in dashed lines in FIG. 11), wherein the outer contour 701 preferably is an octagon when the closure element 700 is arranged in a flat unfolded state. Preferably, the closure element 700 is designed to unfold, particularly like an umbrella, at least partially after being introduced into the borehole, such that the closure element 700 presses against the surrounding wall of the borehole and thus prevents bone cement from dropping out of the borehole. The outer diameter 702 of the closure element 700 preferably corresponds to the outer diameter of the cannula 50 or inner diameter of the borehole.

Further, the closure element 700 is preferably designed to be pushed into the borehole (e.g. of the femoral neck) through the cannula 50 with help of the stamper 60, particularly under X-ray control, before introducing bone cement into the borehole. The diameter of the closure element 700 particularly corresponds to the diameter of the cannula 50.

The closure element 700 can be made out of sterilized Lackmus paper, dyed in standardized Contrast Liquid Solution to make it visible under X-ray control.

Due to Osteoporosis and the individual shape of the femoral neck spongiosa it might happen that bone cement is pushed towards the distal femoral neck area during the femoroplastic procedure, thus provoking a pistol grip shaped bone cement implant.

We suggest that this form gives hypothetically even a higher strength to the femoral neck area and prevents it of pertrochanteric fractures as a side effect of the femoroplastic procedure. The dimensions of FIG. 10 may be chosen to be A=145.00 mm, B=110.00 mm, C=10.00 mm, curvature D=2.00 mm, E=25.50 mm, F=10.00 mm, curvature G=4.00 mm.

It is to be noted that the dimensions of the surgery tool stated above can be adapted to the individual surgical purpose if necessary.

1. The surgical tool for performing surgery on a bone, particularly for reinforcing the bone in a miniinvasive manner, comprising:
a first and a second element extending along a longitudinal axis, respectively, wherein the two elements each comprise a shell defining a hollow space of the respective element, wherein the second element is designed to be inserted into the hollow space of the first element along the first element's longitudinal axis in order to arrange the two elements in an embedded state, in which the two elements are arranged coaxially with respect to each other and in which the shell of the first element encompasses the shell of the second element, wherein particularly the second element being arranged in said embedded state is designed to be pushed with a first free end of the second element ahead towards the bone so as to make contact with said bone,
a guiding element extending along a longitudinal axis, wherein the guiding element comprises a first free end, and wherein the guiding element is designed to be inserted into the hollow space of the second element along the second element's longitudinal axis with said first free end of the guiding element ahead, in order to arrange the guiding element in an inserted state with respect to the second element when the latter is arranged in said embedded state, in which inserted state the guiding element is arranged coaxially with respect to the second element and encompassed by the shell of the second element, and wherein the guiding element is designed to be drilled into said bone with its first free end ahead when being arranged in said inserted state so as to anchor said guiding element in said bone, and
a drill extending along a longitudinal axis, which drill comprises a shell defining a hollow space of the drill, wherein the drill, particularly after having removed the second element from the first element and from the guiding element, is designed to be put over the guiding element along the drill's longitudinal axis with a drill head of the drill ahead in order to arrange the drill in a drilling state with respect to the guiding element and the first element, in which drilling state the first element encompasses said drill for tissue protection and the guiding element is arranged in the hollow space of the drill so as to guide said drill, and wherein the drill head is designed to drill a borehole into the corticalis and the spongiosa of said bone in said drilling state.

2. The surgical tool according to claim 1, characterized in that the second element, particularly after removal of the drill and the first element from the guiding element and re-arrangement of the second element over the guiding element such that the latter is arranged in the hollow space of the second element again, is designed to be inserted with its first free end ahead into said borehole along the second element's longitudinal axis, particularly so as to bridge the corticalis of said bone.

3. The surgical tool according to claim 1, characterized in that the tool further comprises a cannula, which cannula extends along a longitudinal axis, wherein the cannula comprises a shell defining a hollow space of the cannula, through which bone cement can be piped that is to be injected into said borehole, wherein the cannula is designed to be inserted into the hollow space of the second element along the second element's longitudinal axis with a first free end of the cannula ahead in order to arrange the cannula in an injection state with respect to the second element, particularly after the guiding element has been removed from the second element, in which injection state the cannula is arranged coaxially with respect to the second element and encompassed by the shell (200) of the second element.

4. The surgical tool according to claim 3, characterized in that the cannula is designed to protrude with its first free end out of the hollow space of the second element along the second element's longitudinal axis, particularly into the spongiosa of the bone, wherein particularly said first free end of the cannula delimits an injection opening of the cannula, through which bone cement can be discharged out of the hollow space of the cannula.

5. The surgical tool according to claim 3, characterized in that the tool further comprises a stamper extending along a longitudinal axis, wherein the stamper is designed to be inserted into the hollow space of the cannula along the cannula's longitudinal axis with a first free end of the stamper ahead, in order to push bone cement residing in the hollow space of the cannula out of said hollow space, particularly through said borehole into the spongiosa of the bone.

6. The surgical tool according to claim 5, characterized in that the stamper and the cannula are designed to be moved together in the hollow space of the second element along the second element's longitudinal axis from a retracted first position into an advanced second position, particularly so as to push bone cement injected into said borehole into the spongiosa, thereby forming a hollow circular cylindrical bone cement implant in the spongiosa of said bone, wherein particularly said second element is designed to guide the cannula and the stamper upon said movement from the retracted position into the advanced position.

7. The surgical tool according to claim 5, characterized in that the stamper comprises a second free end opposing the first free end along the longitudinal axis of the stamper, wherein the stamper comprises a circular plate at the second free end extending perpendicular to the longitudinal axis of the stamper.

8. The surgical tool according to claim 1, characterized in that the first free end of the second element protrudes along its longitudinal axis out of the first element in the embedded state.

9. The surgical tool according to claim 8, characterized in that the first free end of the second element is rounded.

10. The surgical tool according to claim 1, characterized in that the second element comprises a second free end opposing the first free end of the second element along the longitudinal axis of the second element, wherein particularly said second free end delimits an opening through which said cannula and/or said guiding element can be inserted into the hollow space of the second element.

11. The surgical tool according to claim 10, characterized in that, the second element comprises a portion that broadens towards said second free end, particularly such that said portion comprises at least in sections the shape of a truncated cone.

12. The surgical tool according to claim 11, characterized in that the second free end of the second element forms a circulating protrusion protruding from an outside of said portion.

13. The surgical tool according to claim 1, characterized in that the first free end of the first element is rounded.

14. The surgical tool according to claim 1, characterized in that the first element comprises a second free end opposing the first free end of the first element along its longitudinal axis, wherein particularly said second free end delimits an opening, through which the second element can be inserted into the hollow space of the first element.

15. The surgical tool according to claim 14, characterized in that the hollow space of the first element comprises a section, along which the hollow space of the first element broadens towards said opening of the first element, particularly such that said section of the hollow space of the first element comprises the shape of a truncated cone, particularly such that said section of the hollow space of the first element is able to receive said portion of the second element.

16. The surgical tool according to claim 14, characterized in that the second free end of the first element forms a circulating protrusion protruding from an outside of the shell of the first element.

17. The surgical tool according to claim 1, characterized in that the drill head encompasses an end portion of the hollow space of the drill, which drill head is particularly formed as a spiral drill.

18. The surgical tool according to claim 1, characterized in that the shell of the drill comprises an outside, wherein a plurality of recesses are formed in said outside of the shell of the drill along the longitudinal axis of the drill, wherein particularly the recesses are equidistantly arranged.

19. The surgical tool according to claim 1, characterized in that the drill comprises a second free end opposing the drill head along the longitudinal axis of the drill, wherein the drill comprises a hexagonal cross sectional contour at the second free end, particularly for clamping of the drill in a boring socket of a drilling machine.

20. The surgical tool according to claim 1, characterized in that the tool further comprises a closure element being designed to be introduced into said borehole, particularly so as to retain bone cement that was injected into the borehole.

21. The surgical tool according to claim 20, characterized in that the closure element is designed to be introduced into said borehole in a folded state, wherein the closure element is further designed to unfold at least partially when positioned in said borehole, particularly such that the closure element presses against a surrounding wall of said borehole.

22. The surgical tool according to claim 3, characterized in that the closure element is designed to be pushed into the borehole through the cannula with help of the stamper.

23. The surgical tool according to claim 22, characterized in that the closure element comprises an outer diameter that corresponds to the outer diameter of the cannula.

24. The surgical tool according to claim 21, characterized in that the closure element comprises an outer contour which comprises a serrated shape in said folded state and an octagonal shape in a completely unfolded state.

25. The surgical tool according to claim 20, characterized in that the closure element is made out of a material or comprises a material, particularly paper, particularly sterilized Lackmus paper, wherein said material is designed to be detectable by X-rays, wherein particularly said material is dyed, particularly in standardized contrast liquid solution, so that it can be detected by means of X-rays.
